(19) United States
(12) Patent Application Publication
(10) Pub. No.: US 2018/0147081 A1
(43) Pub. Date: May 31, 2018

(54) Rib Belt

(71) Applicant: Ribcure Co., Ltd., Taichung City (TW)

(72) Inventors: Ying Chieh Su, Tainan City (TW); Yuan-Wei Yu, Tainan City (TW); Cheng-Teng Hsu, Taichung City (TW); Chun Sung Liu, Taichung City (TW)

(21) Appl. No.: 15/820,751

(22) Filed: Nov. 22, 2017

(30) Foreign Application Priority Data
Nov. 29, 2016 (TW) 105139279

(51) Int. Cl. A61F 5/03; A61F 5/02; A61F 13/00; A61F 13/02

(52) U.S. Cl. CPC A61F 5/03; A61F 13/0273; A61F 13/00063; A61F 5/02

(57) ABSTRACT

A rib belt has a dragging belt and two patches. The dragging belt is length adjustable and has two opposite ends. The two patches are respectively connected to the two opposite ends of the dragging belt. Each one of the two patches has an adhesive face. The dragging belt drags the two patches in a stretching manner to resist a contracting force caused by muscle contraction.

### BACKGROUND OF THE INVENTION

### 1. Field of the Invention

The present invention relates to an appliance of orthotics or an appliance of trauma, and more particularly to a rib belt that positions ribs in a stretching manner.

### 2. Description of Related Art

According to medical statistics, 70% of patients suffer from chest contusion in traumatic events. At least 50% of the said patients are diagnosed with fracture of ribs. However, even those patients without definite fracture of ribs suffer from severe pain caused by inflammation of periosteum that covering the ribs. A human being has twelve pairs of ribs. Each one of the ribs is linked to the spine at the posterior end of the rib and connected to the sternum via a costal cartilage at the anterior end of the rib. The ribs form a ribcage. A few rib fractures do not cause deformity of the ribcage. In a common circumstance, the chance of a multiple segmental rib fracture is low. The broken rib is not only restricted by structure of the ribcage but also by the intercostal muscles. Not like a long bone, broken parts of the broken rib do not offset largely to disable a patient from mobility. The main reason that makes the patient difficult to move is the hardly endurable pain.

The patient feels pain when he/she changes position or even just breathes. Muscles of chest wall, intercostals muscles, or the offset of the broken parts of the broken rib drags periosteum covering the ribs to cause the intolerable pain. Therefore, how to alleviate the pain that occurs as the patient is moving and breathing is a main issue, especially for reducing complications.

Conventional treatment to ease the pain caused by fracture of rib includes three orientations:

1. Medication: combination of analgesic drugs and muscle relaxants is commonly applied to ease the pain of the patient suffering from rib fracture, to relax muscles adjacent to the broken rib, and to keep the muscle adjacent to the broken rib free from convulsion. The medication may provide pain relief for the patient at rest. However, the painkilling effect of the medication is quite limited for the patient during movement.

2. External fixation: easing the pain of the patient suffering from rib fracture by lowering the chance of the offset occurring between the broken parts of the broken rib or by fixing the muscles adjacent to the broken rib.

The external fixation includes:

(1) Pressing by bare hand: pressing a position adjacent to the broken rib by the palms or the forearm by the patient himself/herself. However, the aged patient may have little strength for pressing to achieve an ideal effect of pain relief.

(2) Sling: mainly recommended for treating patient with clavicle fracture. The sling may provide pain relief for the patient suffering from rib fracture at the first to sixth broken ribs. However, the sling may interfere with the daily activities of the patient.

(3) Figure-8 bandage: mainly applied to fix a broken clavicle. The figure-8 bandage causes severe pain when initially wrapped on the body of the patient because of dragging the ribs. And the axilla of the patient sweats a lot to cause discomfort.

(4) Rib belt: most common and easy device for fixing broken ribs. The length of the rib belt is adjusted and fixed by a plurality of hook-and-loop fasteners. The rib belt applies a compression force to the chest of the patient to achieve the same effect of the above mentioned pressing by bare hand. U.S. Pat. No. 3,400,710, U.S. Pat. No. 6,516,804, and CN Patent No. 203609559 respectively disclose a device of the rib belt. However, the rib belt restricts expansion of the chest and interferes with the breathing and coughing of the patient suffering from rib fracture. The rib belt may exert an extreme compression force and intensify offset between the broken parts of the broken rib, and may cause severe pain and even bleeding. The updated version of Advanced Trauma Life Support, ATLS does not recommend adapting the rib belt to an aged person.

(5) Rib protector vest: the rib protector vest is a variation of the rib belt. The rib protector vest is fastened on the chest by shoulder harnessing. However, the rib protector vest has defects of high manufacturing expense and muggy feel.

(6) Elastic therapeutic tape: applying elastic and breathable tape to adhere to the skin for stabilizing the subcutaneous tissue, fascia, and muscle beneath the skin to restrict contraction and contracting direction of the muscle. However, the elastic therapeutic tape provides only compression effect, and the performance of painkilling of the elastic therapeutic tape is not good due to the limited tension provided by the elastic therapeutic tape.

(7) Chest orthosis: adhering to the skin for stabilizing the subcutaneous tissue, fascia, and muscle beneath the skin to restrict contraction of muscle and offset of the broken rib. TW Patent No. M317251, U.S. Pat. No. 7,198,609, and U.S. Pat. No. 8,852,132 respectively disclose a device of the chest orthosis. The chest orthosis has a brace to resist the contraction force of the muscle. However, the curvature of the chest orthosis is hard to fit the human body and decreases the effect of resisting contraction force of the muscle.

3. Internal fixation: fixing the ribs by surgery. The pain caused by broken ribs lasts for approximately seven days. The surgery for applying the internal fixation has to detach muscles attached on the ribs. The internal fixation surgery not only causes a large scar, but also diminishes muscular strength of the chest. Therefore, the internal fixation is less acceptable by patients.

The external fixations with compressing fixing manner not only restrict the expansion of the chest and make the patient hard to breathe and cough, but also intensify offset between the broken parts of the broken rib, and cause severe pain and even bleeding.

To overcome the shortcomings of the conventional fixation means, the present invention provides a rib belt to mitigate or obviate the aforementioned problems.

### SUMMARY OF THE INVENTION

The main objective of the present invention is to provide a rib belt that is able to position ribs in a stretching manner.

The rib belt comprises a dragging belt and two patches. The dragging belt is length adjustable and has two opposite ends. The two patches are respectively connected to the two opposite ends of the dragging belt. Each one of the two patches has an adhesive face. The dragging belt drags the two patches in a stretching manner to resist a contracting force caused by muscle contraction.

Other objects, advantages, and novel features of the invention will become more apparent from the following detailed description when taken in conjunction with the accompanying drawings.

### BRIEF DESCRIPTION OF THE DRAWINGS

FIG. 1 is an exploded perspective view of a first embodiment of a rib belt in accordance with the present invention;

FIG. 2 is a schematic side view of the rib belt in FIG. 1, showing the rib belt worn on a patient;

FIG. 3 is an enlarged schematic side view of the rib belt in FIG. 1, showing a patch adhered to a body of the patient;

FIG. 4 is an enlarged schematic side view of the rib belt in FIG. 1, showing another patch adhered to the body of the patient;

FIG. 5 is a schematic top view of the rib belt in FIG. 1;

FIG. 6 is a side view of a patch and an indexing card of the rib belt in accordance with the present invention;

FIG. 7 is an exploded perspective view of a second embodiment of a rib belt in accordance with the present invention;

FIG. 8 is a side view of a third embodiment of a rib belt in accordance with the present invention; and

FIG. 9 is an exploded perspective view of a fourth embodiment of a rib belt in accordance with the present invention.

### DETAILED DESCRIPTION OF PREFERRED EMBODIMENTS

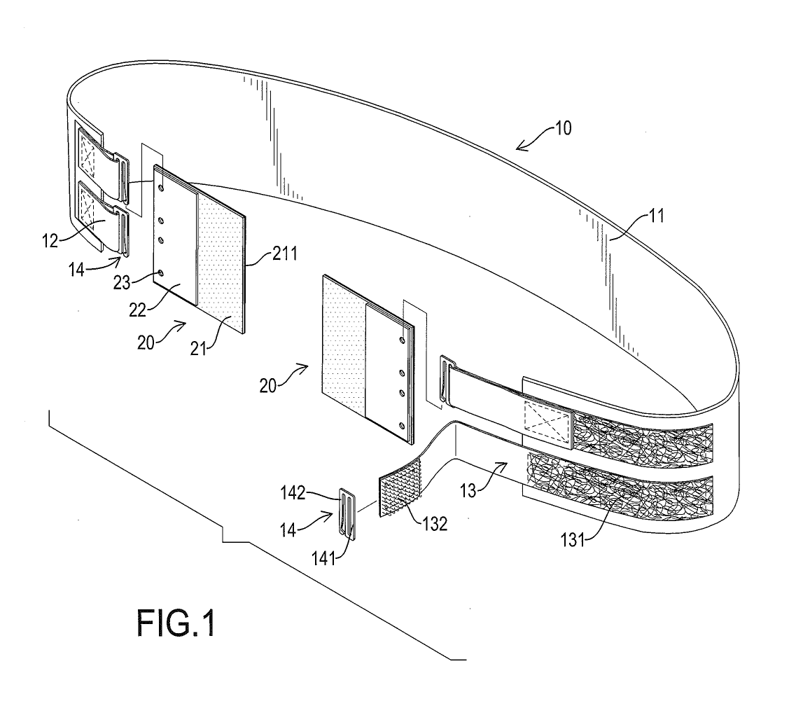

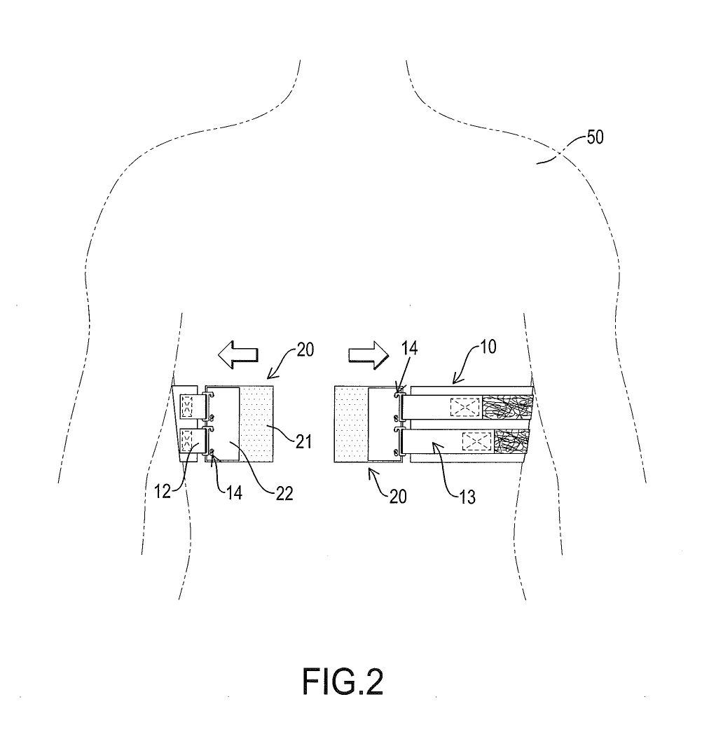

With reference to FIGS. 1 and 2, a first embodiment of a rib belt in accordance with the present invention has a dragging belt 10 and two patches 20. The dragging belt 10 has a belt body 11, two fixed strips 12, two adjustable strips 13, and four fastening units 14. The belt body 11 has a length, a width, and two opposite ends. The length of the belt body 11 is less than a bust of a human body 50. The width of the belt body 11 is about 50 mm to 150 mm. The belt body 11 may be made of nonwoven fabric, gauze, nylon webbing, or an elastic belt. In consideration of hygiene and manufacturing expense, the dragging belt 10 may be a disposal product designed for one-time use. In the first embodiment of the present invention, the belt body 11 is made of nonwoven fabric. The two opposite ends of the belt body 11 are a fixed end and an adjustable end, respectively. The two fixed strips 12 are inflexible, are connected to the fixed end of the belt body 11, and are parallel to and spaced from each other. The two fixed strips 12 are turned up and sewed on the fixed end of the belt body 11.

The two adjustable strips 13 are inflexible, are connected to the adjustable end of the belt body 11, and are parallel to and spaced from each other. Each one of the two adjustable strips 13 has two opposite ends and two hook-and-loop fasteners 131, 132. One of the two opposite ends of each adjustable strip 13 is connected to the adjustable end of the belt body 11 by sewing. The two hook-and-loop fasteners 131, 132 are respectively connected to the two opposite ends of each one of the adjustable strips 13. The two hook-and-loop fasteners 131, 132 are attachable to each other for adjusting a length of the adjustable strip 13.

The four fastening units 14 are respectively connected to the two fixed strips 12 and the two adjustable strips 13. Each one of the four fastening units 14 is a figure-nine hook and has a fastening loop 141 and a hook 142. The fastening hoop 141 is designed for mounting around a corresponding fixed strip 12 or a corresponding adjustable strip 13. The hook 142 is designed for connecting a corresponding patch 20.

Each one of the two patches 20 has a base material 21, a reinforcing material 22, and four connecting holes 23. The base material 21 has an adhesive face 211 covered by release paper. The adhesive face 211 may be further coated with anti-inflammatory and analgesic drugs such as nonsteroid anti-inflammatory drugs (NSAIDs) or morphine to make the patch 20 have an anti-inflammatory and analgesic effect. The reinforcing material 22 is inflexible and is connected to the base material 21. In the first embodiment of the present invention, the base material 21 is made of an elastic bandage. The reinforcing material 22 has to be rigid enough for being connected by two of the four fastening units 14. In the first embodiment of the present invention, the reinforcing material 22 is an inflexible nonwoven fabric. The reinforcing material 22 may be implemented as a coating layer of rubber or plastic materials for connecting the two fastening units 14.

The four connecting holes 23 are formed through the reinforcing material 22 and divided into two pairs corresponding to the two hooks 142 of the two fastening units 14 that are respectively connected to the two fixed strips 12 or the two adjustable strips 13.

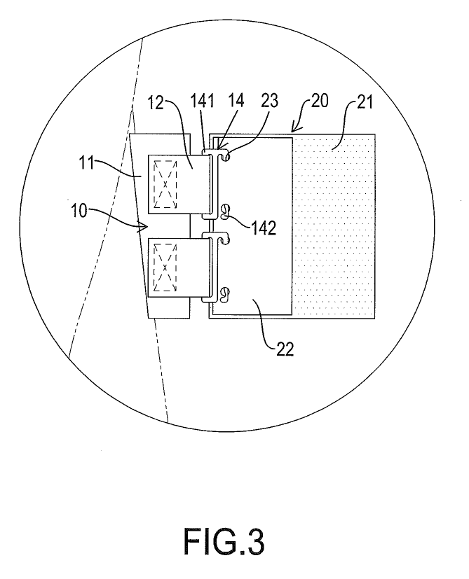

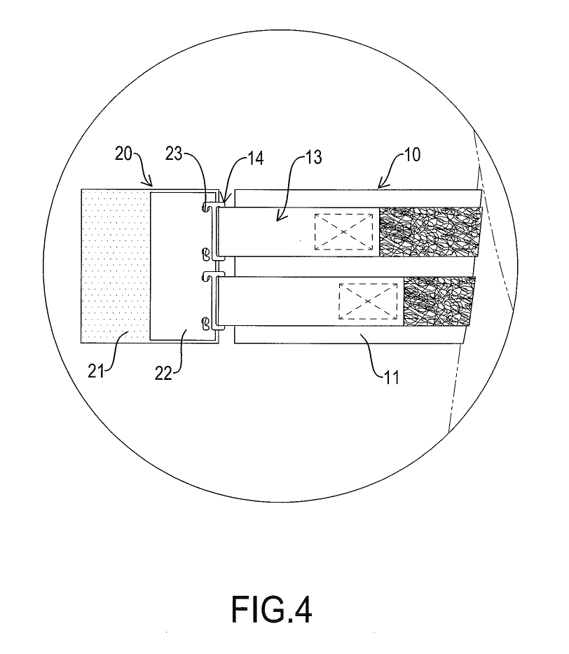

With reference to FIGS. 1 to 4, each one of the four fastening units 14 is inserted through two corresponding connecting holes 23 of one of the two patches 20. One of the two patches 20 is connected to the two fixed strips 12 via two of the four fastening units 14. The other one of the two patches 20 is connected to the two adjustable strips 13 via the other two of the four fastening units 14. When the rib belt in accordance with the present invention is in use, the dragging belt 10 is wrapped on a chest of the human body 50. The two patches 20 are adhered to two sides of a fractured rib portion. The two adjustable strips 13 may be adjusted for dragging the two patches 20 with a suitable stretching force. As the base material 21 of each one of the two patches 20 is flexible, the base material 21 is able to adjust the stretching force to ease the pain when a patient wears the rib belt in accordance with the present invention.

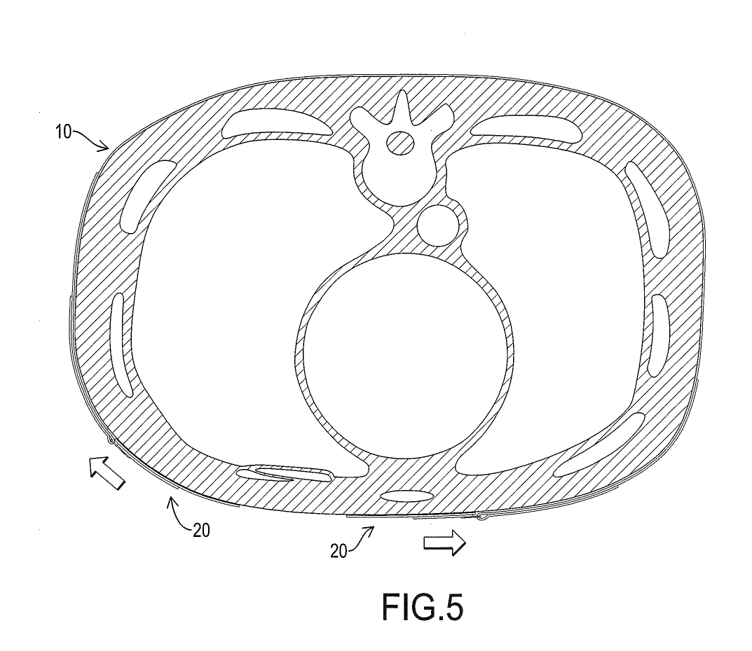

With reference to FIGS. 2 and 5, the rib belt in accordance with the present invention provides the stretching for resisting a contracting force caused by contraction of muscles to avoid pain caused by two broken parts of the fractured rib pressing each other. The rib belt in accordance with the present invention provides the fractured rib with a low tension recovery circumstance and allows the patient to breathe, cough, exercise, or get off the bed without interfering with normal expansion of the chest of the patient.

With reference to FIGS. 1 and 2, each one of the two patches 20 is detachable via two corresponding fastening units 14 and four corresponding connecting holes 23 of the patch 20. The two patches 20 are replaceable for personal hygiene.

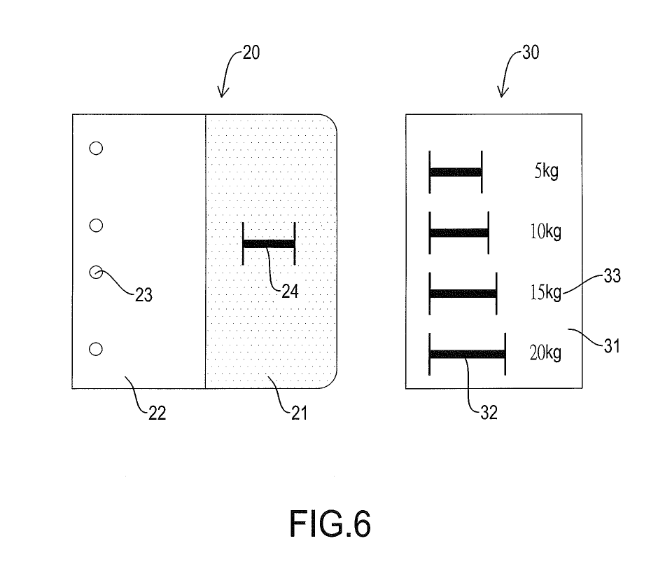

With reference to FIGS. 1 and 6, each one of the two patches 20 may further have an identifying mark 24 that is able to elongate with the base material 21 with flexibility. The rib belt in accordance with the present invention may further have an indexing card 30. The indexing card 30 has a transparent sheet 31, multiple labels 32, and multiple figures 33. The multiple labels 32 of different lengths representing multiple different tensions are disposed on the transparent sheet 31. The multiple figures 33 are disposed on the transparent sheet 31 and respectively aside the multiple labels 32, denoting magnitudes of the multiple tensions. Comparing the identifying mark 24 of one of the two patches 20 with the indexing card 30, the user can readily know the stretching force provided by the dragging belt 10. A doctor or the patient can determine a suitable stretching force by comparing the identifying mark 24 and the indexing card 30.

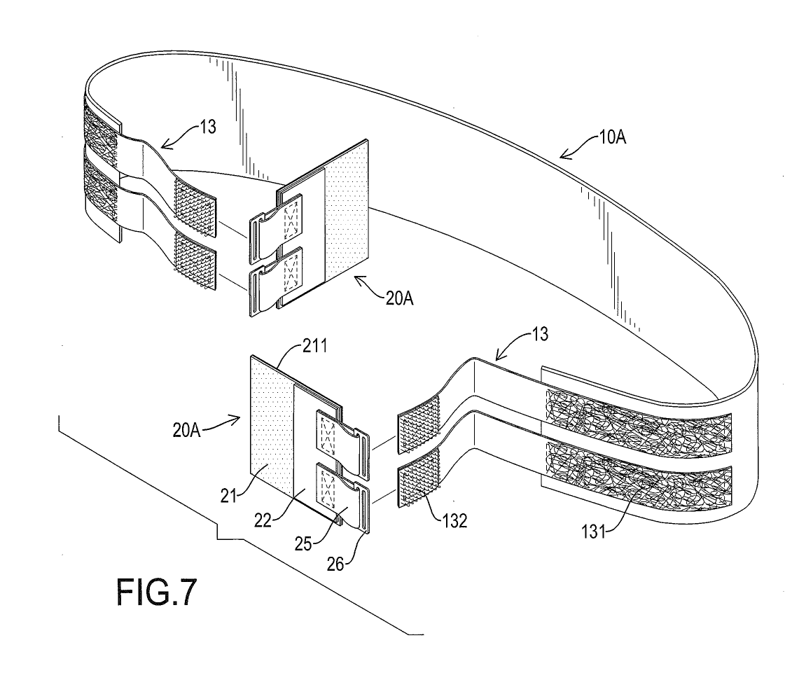

With reference to FIG. 7, the second embodiment of the rib belt in accordance with the present invention has a dragging belt 10A and two patches 20A. The dragging belt 10A has two opposite ends and four adjustable strips 13. Two of the four adjustable strips 13 are connected to one of the two opposite ends of the dragging belt 10A. The other two of the four adjustable strips 13 are connected to the other one of the two opposite ends of the dragging belt 10A. Each one of the four adjustable strips 13 is connected to the dragging belt 10A by sewing and by an adequate connecting length for length adjusting. Each one of the four adjustable strips 13 has two opposite ends and two hook-and-loop fasteners 131, 132 respectively connected to the two opposite ends of the adjustable strip 13. The two hook-and-loop fasteners 131, 132 are attachable to each other. An attaching position of the two hook-and-loop fasteners 131, 132 may be altered for adjusting a length of the adjustable strip 13 that is defined by a corresponding end of the dragging belt 10A.

Each one of the two patches 20 has the base material 21, the reinforcing material 22, two connecting strips 25, and two connecting rings 26. The base material 21 is flexible and has an adhesive face 121. The reinforcing material 22 is connected to the base material 21. The two connecting strips 25 are turned up and sewed on the reinforcing material 22. The two connecting strips 25 connected to the reinforcing material 22 are spaced apart and parallel to each other. The two connecting rings 26 are respectively connected to the two connecting strips 25 for inserting a corresponding adjustable strip 13.

In the second embodiment of the rib belt in accordance with the present invention, the two patches 20A are connected to the two opposite ends of the dragging belt 10A by respectively inserting the four adjustable strips 13 through the four connecting rigs 26. The dragging belt 10A is wrapped on the chest of the human body. The two patches 20A are adhered to the two sides of the fractured rib portion. The four adjustable strips 13 connected to the two opposite ends of the dragging belt 10A may be adjusted for providing a suitable stretching force to drag the two patches 20A. The suitable stretching force provided by the rib belt in accordance with the present invention resists the contracting force caused by contraction of muscles.

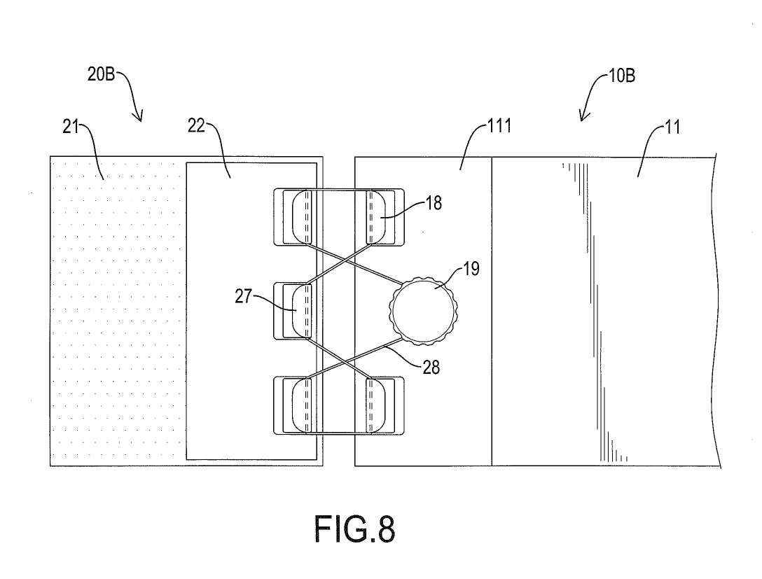

With reference to FIG. 8, the third embodiment of the rib belt in accordance with the present invention has two opposite ends, a dragging belt 10B, and two patches 20B disposed at the two opposite ends of the rib belt, respectively. The two opposite ends of the rib belt are identical. FIG. 8 shows only one of the two opposite ends of the rib belt. For convenience of illustration, only one of the two opposite ends of the rib belt in accordance with the present invention is elaborated hereafter. The dragging belt 10B has two opposite ends and a belt body 11. The belt body 11 has two opposite ends. Each one of the two opposite ends of the belt body 11 is connected to a corresponding one of the two patches 20B. Each one of the two opposite ends of the belt body 11 has a reinforcing sheet 111, and is connected by at least one hooking unit 18. The reinforcing sheet 111 of each one of the two opposite ends of the belt body 11 may be a fabric or a rubber sheet. Each one of the two patches 20B is connected by at least one hooking unit 27. A pull string 28 is wound around the at least one hooking unit 18 connected to one of the two opposite ends of the belt body 11 and the at least one hooking unit 27 connected to the corresponding one of the two patches 20B to connect the end of the belt body 11 and the corresponding one of the two patches 20B.

In the third embodiment of the rib belt in accordance with the present invention, the at least one hooking unit 18 connected to each one of the two opposite ends of the belt body 111 comprises two hooking units 18, and the at least one hooking unit 27 connected to each one of the two patches 20B comprises three hooking units 27. Each one of the two opposite ends of the belt body 11 is further connected by a knob 19 that is rotatable relative to the belt body 11 and disposed between the two hooking units 18 connected to the end of the belt body 11. The pull string 28 wound around the two hooking units 18 and the three hooking units 27 passes through the knob 19 and is connected to the knob 19. The knob 19 is rotated to adjust a distance between each one of the two opposite ends of the belt body 11 and the corresponding one of the two patches 20B. The rotatable knob 19 is capable of adjusting the stretching force provided by the rib belt in accordance with the present invention as well.

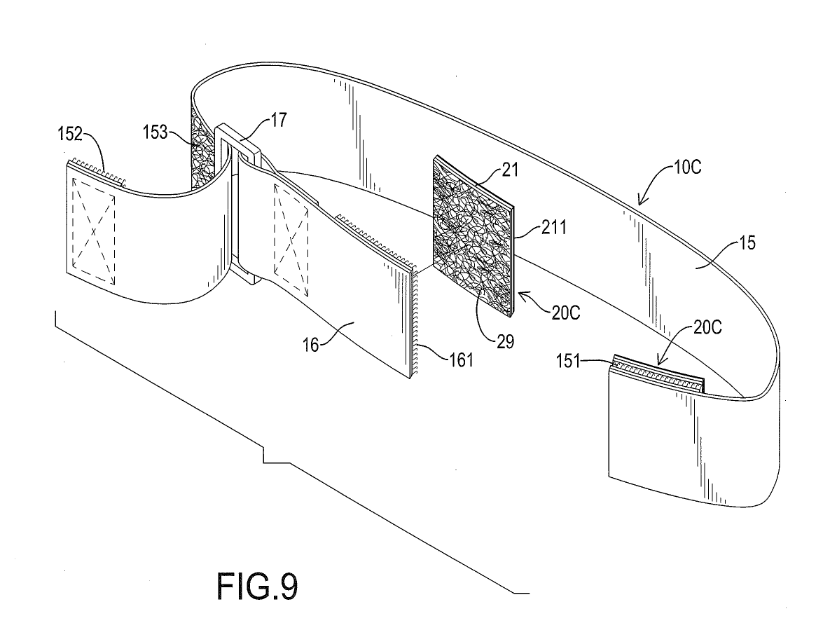

With reference to FIG. 9, the fourth embodiment has a dragging belt 10C and two patches 20C. The dragging belt 10C has an adjustable belt 15 and a connecting belt 16. The adjustable belt 15 has two opposite ends and a hook-and-loop fastener 151 disposed at one of the two opposite ends of the adjustable belt 15. The connecting belt 16 is connected to the other one of the two opposite ends of the adjustable belt 15 and has a hook-and-loop fastener 161. The hook-and-loop fastener 161 is connected to an end of the connecting belt 16 and is distal from the hook-and-loop fastener 151 of the adjustable belt 15. The connecting belt 16 is connected to a buckle 17. The adjustable belt 15 is inserted through the buckle 17 and has two hook-and-loop fasteners 152, 153 that are attachable to each other.

One of the two patches 20C is connected to the connecting belt 16. The other one of the two patches 20C is connected to the adjustable belt 15. Each one of the two patches 20C has the base material 21 and a hook-and-loop fastener 29. The base material 21 is flexible and has the adhesive face for adhering to the human body 50. The hook-and-loop fastener 29 of each one of the two patches 20C is attachable to the hook-and-loop fastener 151 of the adjustable belt 15 and the hook-and-loop fastener 161 of the connecting belt 16. The two patches 20C may be detachably attached to the adjustable belt 15 and the connecting belt 16 by double-sided tapes, hooking units, buttons, or staples.

When the fourth embodiment of the rib belt in accordance with the present invention is in use, the dragging belt 10C is wrapped on the chest of the human body 50. The two patches 20C are adhered to two sides of the fractured rib portion, respectively. The adjustable belt 15 may be adjusted to make the dragging belt 10C provide a suitable stretching force to drag the two patches 20C. The fourth embodiment of the rib belt in accordance with the present invention may also provide the stretching force to resist the contracting force caused by the contraction of muscles.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A rib belt comprising:
a dragging belt being length adjustable and having two opposite ends; and
two patches respectively connected to the two opposite ends of the dragging belt and each one of the two patches having an adhesive face;
the dragging belt dragging the two patches in a stretching manner to resist a contracting force caused by muscle contraction.

2. The rib belt as claimed in claim 1, wherein the two patches are detachably connected to the two opposite ends of the dragging belt.

3. The rib belt as claimed in claim 2, wherein
each one of the two patches has
a base material with flexibility; and
a reinforcing material connected to the base material; and
the adhesive face of the patch is disposed on the base material.

4. The rib belt as claimed in claim 3, wherein the adhesive face is coated with an anti-inflammatory drug.

5. The rib belt as claimed in claim 3, wherein the base material has an indentifying mark that is able to elongate with the base material.

6. The rib belt as claimed in claim 5, wherein the rib belt further has an indexing card having
a transparent sheet;
multiple labels of different lengths, the labels disposed on the transparent sheet and representing multiple different tensions; and
multiple figures disposed on the transparent sheet and respectively aside the multiple labels, respectively denoting magnitudes of the multiple tensions.

7. The rib belt as claimed in claim 1, wherein
the dragging belt has
a belt body with two opposite ends;
at least one fixed strip connected to one of the two opposite ends of the belt body; and
at least one adjustable strip connected to the other one of the two opposite ends of the belt body; and
one of the two patches is connected to the at least one fixed strip, and the other one of the two patches is connected to the at least one adjustable strip.

8. The rib belt as claimed in claim 6, wherein
the dragging belt has
a belt body with two opposite ends;
at least one fixed strip connected to one of the two opposite ends of the belt body; and
at least one adjustable strip connected to the other one of the two opposite ends of the belt body; and
one of the two patches is connected to the at least one fixed strip, and the other one of the two patches is connected to the at least one adjustable strip.

9. The rib belt as claimed in claim 7, wherein
the dragging belt has two fastening units;
one of the two fastening units is connected to the at least one fixed strip and one of the two patches, and the other one of the two fastening units is connected to the at least one adjustable strip and the other one of the two patches;
each one of the two patches has at least one connecting hole; and
each one of the two fastening units has a hook connected to the at least one connecting hole of a patch that is connected to the fastening unit.

10. The rib belt as claimed in claim 8, wherein
the dragging belt has two fastening units;
one of the two fastening units is connected to the at least one fixed strip and one of the two patches, and the other one of the two fastening units is connected to the at least one adjustable strip and the other one of the two patches;
each one of the two patches has at least one connecting hole; and
each one of the two fastening units has a hook connected to the at least one connecting hole of a patch that is connected to the fastening unit.

11. The rib belt as claimed in claim 9, wherein
the at least one fixed strip comprises two fixed strips;
the at least one adjustable strip comprises two adjustable strips; and
each one of the two adjustable strips has two hook-and-loop fasteners respectively disposed at two ends of the adjustable strip and being attachable to each other.

12. The rib belt as claimed in claim 10, wherein
the at least one fixed strip comprises two fixed strips;
the at least one adjustable strip comprises two adjustable strips; and
each one of the two adjustable strips has two hook-and-loop fasteners respectively disposed at two ends of the adjustable strip and being attachable to each other.

13. The belt rib as claimed in claim 1, wherein
the dragging belt has a belt body with two opposite ends;
each one of the two opposite ends of the belt body is connected to at least one adjustable strip;
one of the two patches is connected to the at least one adjustable strip that is connected to one of the two opposite ends of the belt body; and
the other one of the two patches is connected to the at least one adjustable strip that is connected to the other one of the two opposite ends of the belt body.

14. The belt rib as claimed in claim 6, wherein
the dragging belt has a belt body with two opposite ends;
each one of the two opposite ends of the belt body is connected to at least one adjustable strip;
one of the two patches is connected to the at least one adjustable strip that is connected to one of the two opposite ends of the belt body; and
the other one of the two patches is connected to the at least one adjustable strip that is connected to the other one of the two opposite ends of the belt body.

15. The rib belt as claimed in claim 13, wherein one of the two patches that is connected to a corresponding one of the two opposite ends of the belt body is connected to at least one connecting ring that is inserted by the at least one adjustable strip that is connected to the corresponding one of the two opposite ends of the belt body.

16. The rib belt as claimed in claim 14, wherein one of the two patches that is connected to a corresponding one of the two opposite ends of the belt body is connected to at least one connecting ring that is inserted by the at least one adjustable strip that is connected to the corresponding one of the two opposite ends of the belt body.

17. The rib belt as claimed in claim 1, wherein
the dragging belt has an adjustable belt with two opposite ends;
a connecting belt is connected to one of the two opposite ends of the adjustable belt; and
one of the two patches is connected to an end of the connecting belt;
the other one of the two patches is connected to the other one of the two opposite ends of the adjustable belt.

18. The rib belt as claimed in claim 6, wherein
the dragging belt has an adjustable belt with two opposite ends;
a connecting belt is connected to one of the two opposite ends of the adjustable belt; and
one of the two patches is connected to an end of the connecting belt;
the other one of the two patches is connected to the other one of the two opposite ends of the adjustable belt.

19. The rib belt as claimed in claim 17, wherein each one of the two patches is connected to a corresponding one of the two opposite ends of the dragging belt by two hook-and-loop fasteners that are attachable to each other.

20. The rib belt as claimed in claim 18, wherein each one of the two patches is connected to a corresponding one of the two opposite ends of the dragging belt by two hook-and-loop fasteners that are attachable to each other.

21. The rib belt as claimed in claim 19, wherein
the connecting belt is connected to a buckle; and
the adjusting belt is inserted through the buckle and has two hook-and-loop fasteners that are attachable to each other.

22. The rib belt as claimed in claim 20, wherein
the connecting belt is connected to a buckle; and
the adjusting belt is inserted through the buckle and has two hook-and-loop fasteners that are attachable to each other.

23. The rib belt as claimed in claim 1, wherein
the dragging belt has a belt body with two opposite ends respectively connected to the two patches;
each one of the two opposite ends of the belt body is connected by at least one hooking unit;
each one of the two patches is connected by at least one hooking unit; and
a pull string is wound around the at least one hooking unit connected to each one of the two opposite ends of the belt body and the at least one hooking unit connected to a corresponding patch.

24. The rib belt as claimed in claim 6, wherein
the dragging belt has a belt body with two opposite ends respectively connected to the two patches;
each one of the two opposite ends of the belt body is connected by at least one hooking unit;
each one of the two patches is connected by at least one hooking unit; and
a pull string is wound around the at least one hooking unit connected to each one of the two opposite ends of the belt body and the at least one hooking unit connected to a corresponding patch.

25. The rib belt as claimed in claim 23, wherein
the at least one hooking unit connected to each one of the two opposite ends of the belt body comprises two hooking units;
the at least one hooking unit connected to each one of the two patches comprises three hooking units;
each one of the two opposite ends of the belt body is connected by a knob that is rotatable relative to the belt body and disposed between the two hooking units connected to the end of the belt body; and
the pull string wound around the two hooking units connected to the end of the belt body and the three hooking units connected to the corresponding patch passes through and is connected to the knob.

26. The rib belt as claimed in claim 24, wherein
the at least one hooking unit connected to each one of the two opposite ends of the belt body comprises two hooking units;
the at least one hooking unit connected to each one of the two patches comprises three hooking units;
each one of the two opposite ends of the belt body is connected by a knob that is rotatable relative to the belt body and disposed between the two hooking units connected to the end of the belt body; and
the pull string wound around the two hooking units connected to the end of the belt body and the three hooking units connected to the corresponding patch passes through and is connected to the knob.
